(19) United States
(12) Patent Application Publication
(10) Pub. No.: US 2026/0092332 A1
(43) Pub. Date: Apr. 2, 2026

(54) SKIN MICROBIOME-BASED METHODS FOR ACNE ASSESSMENT AND TREATMENT

(71) Applicants: L'OREAL, Paris (FR); The Regents of the University of California, Oakland, CA (US)

(72) Inventors: Amina BOUSLIMANI, Fanwood, NJ (US); Sayeh POURHAMIDI, Aulnay-Sous-Bois (FR); Magali MOREAU, Clark, NJ (US); Jolanta IDKOWIAK-BALDYS, Allendale, NJ (US); Susi DAL BELO, Aulnay-Sous-Bois (FR); Se Jin SONG, Boulder, CO (US); Tyler MYERS, La Jolla, CA (US); Andrew BARTKO, San Diego, CA (US)

(73) Assignees: L'OREAL, Paris (FR); The Regents of the University of California, Oakland, CA (US)

(21) Appl. No.: 19/338,761

(22) Filed: Sep. 24, 2025

Related U.S. Application Data
(60) Provisional application No. 63/701,804, filed on Oct. 1, 2024.

(30) Foreign Application Priority Data
Nov. 12, 2024 (FR) 2412336

(51) Int. Cl. C12Q 1/689

(52) U.S. Cl. CPC C12Q 1/689; C12Q 2600/106; C12Q 2600/158

(57) ABSTRACT

Methods for identifying an individual having a predisposition to developing acne, for assessing severity of acne lesions, and for personalized prevention, diagnosis, and treatment of acne are disclosed. The methods include: (a) obtaining microbiome sample from skin of an individual; (b) determining an amount of one or more bacteria in the microbiome sample, wherein the one or more bacteria are selected from Lawsonella, Cutibacterium, Staphylococcus, Corynebacterium, Streptococcus, Micrococcus, and Neisseriaceae; (c) comparing the amount of the one or more bacteria to a control; and (d) identifying a difference between the amount of the one or more bacteria in the microbiome sample and the control.

### CROSS-REFERENCE TO RELATED APPLICATIONS

This application claims benefit of U.S. Provisional Application No. 63/701,804, filed Oct. 1, 2024, and benefit of French Application No. FR 2412336, filed on Nov. 12, 2024, which are incorporated herein by reference in their entirety.

### FIELD OF THE DISCLOSURE

The present disclosure pertains to methods for evaluating an individual's susceptibility to acne and assessing the severity of acne lesions by analyzing the individual's skin microbiome. It also pertains to methods for preventing or treating acne, including personalized methods based on an individual's unique skin microbiome.

### BACKGROUND

Acne vulgaris, a prevalent dermatological condition affecting millions globally, is the result of a multifaceted interplay among hormonal fluctuations, genetic predispositions, and lifestyle factors. A critical yet frequently underexplored element in acne pathology is the skin microbiome. This complex consortium of microorganisms, encompassing bacteria, fungi, and viruses, resides on and within the skin and plays a substantial role in skin health and disease. A comprehensive understanding of the skin microbiome's influence on acne can lead to innovative strategies for prevention and treatment.

The skin microbiome represents a dynamic ecosystem where diverse microbial populations coexist and interact with each other and the host's immune system. This microbial balance is essential for maintaining skin homeostasis and preventing disease. Specific microbes have been implicated in acne pathogenesis. For instance, Cutibacterium acnes (formerly Propionibacterium acnes) is a Gram-positive, anaerobic bacterium that flourishes in the sebum-rich environments of hair follicles. Although it is normally part of the skin's microbiota, an overgrowth or imbalance of C. acnes can contribute to acne formation. This bacterium secretes enzymes and metabolites that can induce inflammation and lead to follicular obstruction.

Another notable microbial player is the genus Malassezia, a type of yeast normally present in the skin microbiome. Certain species of Malassezia have been linked to acne and other dermatological disorders. These fungi can exacerbate inflammation and skin irritation through the production of specific lipases and other bioactive compounds.

The onset of acne is commonly associated with increased sebum production, which creates a nutrient-rich environment conducive to C. acnes proliferation. Excess sebum can result in follicular occlusion, trapping bacteria and dead skin cells and generating an anaerobic environment that promotes C. acnes growth and acne lesion formation. The overgrowth of C. acnes and other microorganisms can provoke an inflammatory response, as C. acnes produces various molecules that stimulate the immune system, leading to redness, swelling, and pustule formation. The interaction between C. acnes and the host's immune system is pivotal in determining the severity of acne lesions.

Additionally, dietary factors, such as high-glycemic diets and dairy consumption, have been shown to affect sebum production and the skin microbiome, potentially exacerbating acne. Skincare products and antibiotics can also influence the skin microbiome. While antibiotics may reduce C. acnes populations, they can disrupt the balance of beneficial microorganisms, possibly resulting in a rebound effect that aggravates acne. Furthermore, stress has been observed to impact acne development by altering hormone levels and sebum production, which in turn affects the skin microbiome and contributes to acne flare-ups.

Given the significant role of the skin microbiome in acne development and severity—through mechanisms such as sebum production, inflammation, and microbial balance—there is a substantial need for further research into this area. Such research could facilitate the development of novel therapeutic approaches targeting the microbiome to improve acne management and skin health. Advancing our understanding of these interactions will ultimately lead to more effective treatments and preventive measures, enhancing outcomes for individuals affected by this prevalent condition.

### SUMMARY OF THE DISCLOSURE

The present disclosure pertains to methods for evaluating an individual's susceptibility to acne and assessing the severity of acne lesions through the analysis of the skin microbiome. It also includes methods for preventing or treating acne, with a focus on personalized approaches tailored to an individual's unique skin microbiome. Traditionally, acne is addressed only after it manifests, due to the lack of methods for predicting susceptibility. Consequently, acne treatment has largely been a remedial, one-size-fits-all approach. The inventors have identified a personalized strategy that considers the specific composition of their skin microbiome. This targeted approach addresses the underlying causes of acne more precisely, leading to improved treatment outcomes. By analyzing and understanding an individual's unique microbial environment, the methods specifically target dysbiosis (imbalance in the microbiome) that may contribute to acne, rather than treating symptoms alone. Frequency and severity of acne breakouts are reduced or eliminated.

The instant disclosure is drawn to a method for identifying and optionally treating an individual with a predisposition to developing acne. The method comprises:
(a) obtaining microbiome sample from skin of an individual;
(b) determining an amount of one or more bacteria in the microbiome sample, wherein the one or more bacteria are selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof;
(c) comparing the amount of the bacteria to a control;
(d) identifying a difference between the amount of the one or more bacteria in the microbiome sample and the control with respect to at least one bacteria selected from Lawsonella, Cutibacterium, Staphylococcus, Corynebacterium, Streptococcus, and Micrococcus, and Neisseriaceae, preferably wherein the at least one bacteria include Lawsonella, Cutibacterium, or a combination thereof; and
(c) identifying the individual having a predisposition to developing acne based on the difference.

The microbiome sample is from skin that is prone to develop acne, for example, skin of the face, skin of the back, and skin of the shoulders.

The microbiome sample is evaluated to determine the amount of the bacteria in the microbiome sample. In particular, the microbiome sample is evaluated to determine the amount of Lawsonella, Cutibacterium, Staphylococcus, Corynebacterium, Streptococcus, Micrococcus, and/or Neisseriaceae. In many cases, the amount of more than one bacteria is evaluated. For example, the amount of two or more, three or more, four or more, five or more, six or more, or all bacteria are evaluated from the group consisting of Lawsonella, Cutibacterium, Staphylococcus, Corynebacterium, Streptococcus, and Micrococcus, and Neisseriaceae.

The control is typically the amount of bacteria in a microbiome sample from an individual or a population of individuals that are not suffering from acne or are not prone to developing acne. Comparing the amount of the microbiome sample from the skin of an individual prone to developing acne with a control allows for detection of difference. For example, an excess of abundance of one or more of the bacteria or an absence of abundance of one or more of the bacteria indicates that the individual is prone to developing acne.

An individual identified as prone to develop acne may be treated to prevent development of acne or reduce the severity of acne lesions. The treatment involves minimizing or eliminating differences between the abundance of bacteria in the microbiome of the individual's skin and the amount of the bacteria in the control. If the abundance of a particular bacteria in the microbiome of the individual's skin is higher than the control, the individual is treated to reduce the abundance of the particular bacteria. If the amount of a particular bacteria is lower than the control, the individual is treated to increase the abundance of the particular bacteria.

Treatments that minimize differences between the microbiome of the individual's skin and the control include topical treatments, oral treatments, physical treatments, and energy-based treatments. The treatment serves to align the amount of bacteria in the microbiome of the individual's skin with the control. For example, in a preferred embodiment, the treatment comprises topically administering to the individual's skin a pharmaceutical or cosmetic composition. The pharmaceutical or cosmetic composition minimizes the difference between the amount of the bacterial in the microbiome of the individual's skin and the control. Typically, the pharmaceutical or cosmetic composition includes one or more anti-acne compounds. Nonlimiting examples of anti-acne compounds include retinoids, anti-bacterial compounds, anti-inflammatory compounds, essential oils, botanical extracts, probiotics, prebiotics, antibiotics, sulfur, vitamins, and benzoyl peroxide.

In another embodiment, the treatment comprises orally administering one or more active compounds to the individual. Nonlimiting examples of active compounds that may be orally administered to the individual include retinoids antibiotics, hormones, probiotics, prebiotics, omega-3-fatty acids, and vitamins.

In a further embodiment, the treatment comprises administering an energy-based treatment to the individual's skin, for example, light therapy. Nonlimiting examples of light therapy include blue light therapy, red light therapy, yellow light therapy, green light therapy, intense pulsed light therapy, photodynamic therapy, UV light therapy, and infrared therapy.

The amount of bacteria in the microbiome sample can be determined by detecting the nucleic acid of bacteria in the sample. This allows for quantification of bacterial population based on genetic material. Nonlimiting examples of techniques for detecting bacterial nucleic acids include polymerase chain reaction (PCR), 16S rRNA gene sequencing, metagenomic sequencing, and fluorescent in situ hybridization (FISH). In a preferred embodiment, the technique is 16S rRNA gene sequence or metagenomic sequencing. Furthermore, the amount of bacteria can be monitored over time to track changes in bacterial populations in response to treatment and, if needed, the treatment can be updated.

The instant disclosure is also drawn to a method for determining severity of acne lesions for an individual suffering from acne lesions, the method comprising:
(a) obtaining microbiome sample from skin of the individual suffering from acne lesions;
(b) determining an amount of one or more bacteria in the microbiome sample, wherein the one or more bacteria are selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof;
(c) comparing the amount of the bacteria to a control;
(d) identifying how the amount of the one or more bacteria in the microbiome sample differs from the control with respect to abundance of at least one bacteria selected from Lawsonella, Cutibacterium, Staphylococcus, Corynebacterium, Streptococcus, Micrococcus, and Neisseriaceae, preferably wherein the at least one bacteria include Lawsonella, Cutibacterium, or a combination thereof; and
(e) and determining the severity acne lesions based on the difference.

The microbiome sample is from skin suffering from acne lesions. The microbiome sample is evaluated to determine the amount of the bacterial in the microbiome sample. In particular, the microbiome sample is evaluated to determine the amount of Lawsonella, Cutibacterium, Staphylococcus, Corynebacterium, Streptococcus, Micrococcus, and/or Neisseriaceae. In many cases, the amount of more than one bacteria is evaluated. For example, the amount of two or more, three or more, four or more, five or more, six or more, or all bacteria are evaluated from the group consisting of Lawsonella, Cutibacterium, Staphylococcus, Corynebacterium, Streptococcus, Micrococcus, and Neisseriaceae.

The control is typically the amount of bacteria in a microbiome sample from an individual or a population of individuals that are not suffering from acne or are not prone to developing acne. Comparing the amount of the microbiome sample from the skin of an individual suffering from acne with a control allows for detection of difference in amounts of bacteria between the microbiome sample from the skin of the individual suffering from acne and the control. For example, an excess of abundance of one or more of the bacteria or an absence of abundance of one or more of the bacteria elucidates the cause of the acne and how to treat the acne.

The individual suffering from acne may be treated to prevent development of further acne or to reduce the severity of acne lesions. The treatment involves minimizing or eliminating differences between the abundance of bacteria in the microbiome of the individual's skin and the abundance of the bacteria in the control. If the abundance of a particular bacteria in the microbiome of the individual's skin is higher than the control for a particular bacteria, the individual is treated to reduce the abundance of the particular bacteria. If the amount of a particular bacteria is lower than the control, the individual is treated to increase the abundance of the particular bacteria.

Treatments that minimize differences between the microbiome of the individual's skin and the control include topical treatments, oral treatments, physical treatments, and energy-based treatments. The treatment serves to align the amounts of bacteria in the microbiome of the individual's skin with the control. For example, in a preferred embodiment, the treatment comprises topically administering to the individual's skin a pharmaceutical or cosmetic composition that minimizes the difference between the amount of the bacterial in the microbiome of the individual's skin and the control. The pharmaceutical or cosmetic composition includes one or more anti-acne compounds. Nonlimiting examples of anti-acne compounds include retinoids, anti-bacterial compounds, anti-inflammatory compounds, essential oils, botanical extracts, probiotics, prebiotics, antibiotics, sulfur, vitamins, and benzoyl peroxide.

In another embodiment, the treatment comprises orally administering one or more active compounds to the individual, wherein the one or more active compounds are compounds that minimize the difference between the amount of the bacterial in the microbiome of the individual's skin and the control. Nonlimiting examples of active compounds that may be orally administered to the individual include retinoids antibiotics, hormones, probiotics, prebiotics, omega-3-fatty acids, and vitamins.

In a further embodiment, the treatment comprises administering an energy-based treatment to the individual's skin, for example, light therapy. Nonlimiting examples of light therapy include blue light therapy, red light therapy, yellow light therapy, green light therapy, intense pulsed light therapy, photodynamic therapy, UV light therapy, and infrared therapy).

The amount of bacteria in the microbiome sample can be determined by detecting the nucleic acid of bacteria in the sample. This allows for quantification of bacterial populations based on their genetic material. Nonlimiting examples of technique for detecting bacterial nucleic acids include quantitative polymerase chain reaction (qPCR), 16S rRNA gene sequencing, metagenomic sequencing, and fluorescent in situ hybridization (FISH). In a preferred embodiment, the technique is 16S rRNA gene sequencing or metagenomic sequencing. Furthermore, the amount of bacteria can be monitored over time to track changes in bacterial populations in response to treatment.

The instant disclosure is drawn to a method for personalized prevention and treatment of acne comprising:
(a) obtaining microbiome sample from skin of an individual;
(b) determining an amount of one or more bacteria in the microbiome sample, wherein the one or more bacteria are selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof;
(c) comparing the amount of the one or more bacteria to a control;
(d) identifying a difference between the amount of the bacteria in the microbiome sample and the control with respect to at least one bacteria selected from Lawsonella, Cutibacterium, Staphylococcus, Corynebacterium, Streptococcus, Micrococcus, and Neisseriaceae; and
(c) treating the individual by minimizing the difference between the amount of the bacteria in the microbiome of the individual's skin and the control.

The microbiome sample is from skin suffering from acne lesions. The microbiome sample is evaluated to determine the amount of the bacterial in the microbiome sample. In particular, the microbiome sample is evaluated to determine the amount of Lawsonella, Cutibacterium, Staphylococcus, Corynebacterium, Streptococcus, Micrococcus, and/or Neisseriaceae. In many cases, the amount of more than one bacteria is evaluated. For example, the amount of two or more, three or more, four or more, five or more, six or more, or all bacteria are evaluated from the group consisting of Lawsonella, Cutibacterium, Staphylococcus, Corynebacterium, Streptococcus, Micrococcus, and Neisseriaceae.

The control is typically the amount of bacteria in a microbiome sample from an individual or a population of individuals that are not suffering from acne or are not prone to developing acne. Comparing the amount of the microbiome sample from the skin of an individual suffering from acne with a control allows for detection of differences in amount of bacteria between the microbiome sample from the skin of the individual suffering from acne and the control. For example, an excess of abundance of one or more of the bacteria or an absence of abundance of one or more of the bacteria elucidates the cause of the acne and how to treat the acne.

The individual suffering from acne may be treated to prevent development of further acne or to reduce the severity of acne lesions. The treatment involves minimizing or eliminating differences between the abundance of bacteria in the microbiome of the individual's skin and the abundance of the bacteria in the control. If the abundance of a particular bacteria in the microbiome of the individual's skin is higher than the control for a particular bacteria, the individual is treated to reduce the abundance of the particular bacteria. If the amount of a particular bacteria is lower than the control, the individual is treated to increase the abundance of the particular bacteria.

Treatments that minimize differences between the microbiome of the individual's skin and the control include topical treatments, oral treatments, physical treatments, and energy-based treatments. The treatment serves to align the amount of bacteria in the microbiome of the individual's skin with the control. For example, in a preferred embodiment, the treatment comprises topically administering to the individual's skin a pharmaceutical or cosmetic composition that minimizes the difference between the amount of the bacterial in the microbiome of the individual's skin and the control. The pharmaceutical or cosmetic composition includes one or more anti-acne compounds. Nonlimiting examples of anti-acne compounds include retinoids, anti-bacterial compounds, anti-inflammatory compounds, essential oils, botanical extracts, probiotics, prebiotics, antibiotics, sulfur, vitamins, and benzoyl peroxide.

In another embodiment, the treatment comprises orally administering one or more active compounds to the individual, wherein the one or more active compounds are compounds that minimizes the difference between the amount of the bacterial in the microbiome of the individual's skin and the control. Nonlimiting examples of active compounds that may be orally administered to the individual include retinoids antibiotics, hormones, probiotics, prebiotics, omega-3-fatty acids, and vitamins.

In a further embodiment, the treatment comprises administering an energy-based treatment to the individual's skin, for example, light therapy. Nonlimiting examples of light therapy include blue light therapy, red light therapy, yellow light therapy, green light therapy, intense pulsed light therapy, photodynamic therapy, UV light therapy, and infrared therapy).

The amount of bacteria in the microbiome sample can be determined by detecting the nucleic acid of bacteria in the sample. This allows for quantification of bacterial populations based on their genetic material. Nonlimiting examples of technique for detecting bacterial nucleic acids include quantitative polymerase chain reaction (qPCR), 16S rRNA gene sequencing, metagenomic sequencing, and fluorescent in situ hybridization (FISH). In a preferred embodiment, the technique is 16S rRNA gene sequencing or metagenomic sequencing. Furthermore, the amount of bacteria can be monitored over time to track changes in bacterial populations in response to treatment.

### BRIEF DESCRIPTION OF THE DRAWINGS

Implementation of the present technology is described, by way of example only, with reference to the attached figures, wherein:

FIG. 1 shows the various zones on the face of an individual used for collecting microbiome samples from the individual and an outline of a process for obtaining microbiome data;

FIG. 2 shows bacterial profiles collected over 28 days from individuals suffering from acne and individuals not suffering from acne, based on Robust Aitchison Principal Component Analysis (RPCA) beta diversity analysis of data generated using primers that targeted the 16S rRNA gene variable regions (A) (V1-V3); and (B) V4;

FIG. 3 shows improved separation between bacterial profiles collected over 28 days from individuals suffering from acne and individuals not suffering from acne, using Compositional Tensor Factorization (CTF) analysis of data generated with primers that targeted the 16S rRNA gene variable regions (A) (V1-V3) and (B) V4;

FIG. 4 shows similar bacterial alpha diversity (Shannon Entropy measure) between microbiome samples of acne lesional areas from individuals suffering from acne and microbiome samples of non-lesional areas from the individuals suffering from acne, observed with sequence data of variable regions: (A) (V1-V3); and (B) V4;

FIG. 5 shows top bacterial markers associated with acne status identified using the Compositional Tensor Factorization (CTF) analysis from the microbiome samples of acne lesions of individuals suffering from acne and the microbiome samples of non-lesion areas of the individuals suffering from acne. Similar markers were identified using sequence data of variable regions (V1-V3) and V4;

FIG. 6 shows the robust centered log-ratio (RCLR) transformed abundance of bacterial markers identified in acne (lesional, non-lesional) and healthy samples;

FIG. 7 shows that acne markers for Lawsonella and Cutibacterium display an inverse correlation with local lesion severity globally, i.e., their abundance decreases with increasing local lesion severity;

FIG. 8 is a heatmap showing that abundance of bacteria, such as Cutibacterium, Lawsonella, Neisseriaceae correlate with lesion progression over time; and

FIG. 9 shows bacterial profiles (relative abundance of taxa) from skin samples collected from Right and Left cheeks of healthy individuals, which may be considered a control, and from Right and Left cheeks of acne individuals.

The various aspects of the disclosure are not limited to the results, arrangements, and representations shown in the drawings.

### DETAILED DESCRIPTION OF THE DISCLOSURE

Acne vulgaris is a common skin condition that affects millions globally characterized by the presence of comedones, papules, pustules, and sometimes cysts. Traditionally, acne has been attributed to factors such as increased sebum production, androgenic hormones, and the proliferation of Propionibacterium acnes (now known as Cutibacterium acnes). The skin microbiome is a dynamic ecosystem composed of bacteria, fungi, viruses, and archaea. The composition of the skin microbiome varies by body site, age, sex, and environmental factors. Under normal conditions, the skin microbiome contributes to skin homeostasis by protecting against pathogenic microorganisms, modulating immune responses, and maintaining the integrity of the skin barrier. Disruption in the balance of bacteria in the skin's microbiota signals a propensity to develop acne.

By studying the interplay between the skin's microbiota and acne, focusing on microbial composition and dysbiosis, the inventor identified factors contributing to acne pathogenesis. In particular, the inventors discovered specific shifts in the skin microbiome are useful for identifying individuals having a propensity for developing acne and developed methods for preventing and treating acne. By analyzing and understanding an individual's unique microbial environment, the methods specifically target dysbiosis (imbalance in the microbiome) rather than merely treating symptoms post facto. Frequency and severity of acne breakouts are reduced or eliminated.

The inventors identified specific bacteria and imbalances in the bacterial populations (dysbiosis) that are associated with acne and with the severity of acne lesions. These bacteria include Lawsonella, Cutibacterium, Staphylococcus, Corynebacterium, Streptococcus, Micrococcus, and Neisseriaceae.

Lawsonella is a genus of bacteria classified within the phylum Bacteroidota. Taxonomically, it belongs to the Domain: Bacteria, Phylum: Bacteroidota, Class: Bacteroidia, Order: Bacteroidales, Family: Bacteroidaceae, and Genus: Lawsonella. A notable species is Lawsonella clevelandensis, initially isolated from the oral cavity and linked to periodontal disease. These bacteria are Gram-negative, characterized by a thin peptidoglycan layer encased in an outer membrane rich in lipopolysaccharides.

Cutibacterium is a genus of bacteria that encompasses species commonly found in the human skin microbiota. Previously known as Propionibacterium, it belongs to the Domain: Bacteria, Phylum: Actinomycetota, Class: Actinomycetia, Order: Actinomycetales, Family: Propionibacteriaceae, and Genus: Cutibacterium. Members of this genus are Gram-positive, non-motile, anaerobic rods that can appear singly or in clusters and display a pleomorphic (variable shape) morphology under the microscope. These bacteria thrive in low-oxygen environments and play a crucial role in skin health by competing with pathogenic microorganisms, breaking down sebum, and influencing local immune responses. The species, Cutibacterium acnes (formerly Propionibacterium acnes), is a major contributor to acne, colonizing hair follicles and promoting inflammation that leads to acne lesions. Another notable species include Cutibacterium granulosum. In a preferred embodiment, the species Cutibacterium granulosum is included in the one or more bacteria that is assessed and considered in the methods of the instant case.

Staphylococcus is a genus of bacteria notable for its spherical shape and tendency to form clusters resembling grapes. These Gram-positive cocci are prevalent in both the environment and human microbiota. Taxonomically, Staphylococcus belongs to the Domain: Bacteria, Phylum: Firmicutes, Class: Bacilli, Order: Bacillales, Family: Staphylococcaceae, and Genus: Staphylococcus. Prominent species include Staphylococcus aureus, Staphylococcus epidermidis, Staphylococcus saprophyticus, and Staphylococcus lugdunensis. These bacteria form clusters and are facultative anaerobes. S. epidermidis, in particular, is a normal resident of the skin and mucous membranes, contributing to the balance of the skin microbiome and preventing the colonization of more harmful pathogens.

Corynebacterium is a genus comprising both benign and pathogenic species. These Gram-positive rods are distinguished by their club-shaped morphology and often form “V” or “L” shapes in culture. Corynebacterium falls within the Domain: Bacteria, Phylum: Actinomycetota, Class: Actinomycetia, Order: Actinomycetales, Family: Corynebacteriaceae, and Genus: Corynebacterium. Notable species include Corynebacterium diphtheriae, Corynebacterium jeikeium, Corynebacterium urealyticum, and Corynebacterium tuberculostearicum. Corynebacteria are characterized by their distinctive club-shaped appearance and angular arrangements, sometimes resembling “Chinese letters.” Many species, such as C. tuberculostearicum, are part of the normal skin flora and mucous membranes, playing a role in maintaining microbial balance.

Streptococcus is a genus of Gram-positive cocci responsible for a wide range of infections, from mild throat infections to severe systemic diseases. It belongs to the Domain: Bacteria, Phylum: Firmicutes, Class: Bacilli, Order: Lactobacillales, Family: Streptococcaceae, and Genus: Streptococcus. The genus is characterized by its spherical cells that typically form chains or pairs. Key species include Streptococcus pyogenes (Group A Streptococcus), Streptococcus agalactiae (Group B Streptococcus), Streptococcus pneumoniae, Streptococcus viridans (Viridans Streptococci), and Streptococcus bovis.

Micrococcus is a genus of Gram-positive, cocci-shaped bacteria generally considered non-pathogenic. It belongs to the Domain: Bacteria, Phylum: Actinomycetota, Class: Actinomycetia, Order: Micrococcales, Family: Micrococcaceae, and Genus: Micrococcus. Species include Micrococcus luteus, Micrococcus roseus, and Micrococcus varians. These bacteria are typically found in the environment and as part of the normal human skin flora.

Neisseriaceae is a family of bacteria within the order Neisseriales. The most prominent genera in this family are Neisseria and Kingella. Taxonomically, Neisseriaceae belongs to the Domain: Bacteria, Phylum: Proteobacteria, Class: Betaproteobacteria, Order: Neisseriales, and Family: Neisseriaceae. Notable genera include Neisseria and Kingella. Key species of Neisseria include Neisseria meningitidis, Neisseria gonorrhoeae, Neisseria lactamica, Neisseria sicca, and Neisseria subflava. Species of Kingella include Kingella kingae and Kingella denticola.

The instant disclosure pertains to a method for identifying and optionally treating an individual with a predisposition to developing acne. The method comprises:
(a) obtaining microbiome sample from skin of an individual;
(b) determining an amount of one or more bacteria in the microbiome sample, wherein the one or more bacteria are selected from Lawsonella, Cutibacterium, Staphylococcus, Corynebacterium, Streptococcus, and Micrococcus, and Neisseriaceae, preferably wherein the one or more bacteria include Lawsonella, Cutibacterium, or a combination thereof;
(c) comparing the amount of the bacteria to a control;
(d) identifying a difference between the amount of the one or more bacteria in the microbiome sample and the control with respect to at least one bacteria selected from Lawsonella, Cutibacterium, Staphylococcus, Corynebacterium, Streptococcus, Micrococcus, and Neisseriaceae, preferably wherein the at least one bacteria include Lawsonella, Cutibacterium, or a combination thereof; and
(c) identifying the individual having a predisposition to developing acne based on the difference.

The microbiome sample is from skin that is prone to develop acne, for example, skin of the face, skin of the back, and skin of the shoulders.

The microbiome sample is evaluated to determine the amount of the bacteria in the microbiome sample, and the amount of the bacteria is quantified. In particular, the microbiome sample is evaluated to determine the amount of one or more bacteria selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof. In many cases, the amount of more than one bacteria is evaluated. For example, the amount of two or more, three or more, four or more, five or more, six or more, or all bacteria are evaluated, wherein the bacteria are selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof.

Determining an amount of one or more bacteria in the microbiome sample involves quantifying the amount of the bacteria. The quantification of bacteria can be expressed in several ways, including absolute abundance, relative abundance, and through various log-ratio transformations. Absolute abundance refers to the total number of bacterial cells present in the sample, offering a direct count without comparison to other components. This measure provides a straightforward assessment of bacterial load. Relative abundance, on the other hand, calculates the proportion of each bacterial species relative to the total bacterial community in the sample. This approach is useful for understanding the composition of bacterial communities and the relative contributions of different species to the overall microbial landscape.

Preferably, log-ratio transformations are employed to account for data compositionality (i.e. the abundance of any one feature is only interpretable relative to another). Examples of log-ratio transformations include the robust centered log-ratio (RCLR) transformation, centered log-ratio (CLR) transformation, additive log-ratio (ALR) transformation, isometric log-ratio (ILR) transformation, and multi-additive log-ratio (MALR) transformation. Among these, the robust centered log-ratio (RCLR) transformation is preferred.

Centered log-ratio (CLR) transformation normalizes the data by dividing each component by the geometric mean of all components and then applying a logarithmic transformation. This method is particularly effective in minimizing the impact of outliers and deviations from normality, thereby providing a robust approach to analyzing bacterial abundance in microbiome samples. The related robust centered log-ratio (RCLR) transformation uses only the non-zero components.

Additive log-ratio (ALR) transformation involves comparing each component to a reference component, transforming the data into a series of log-ratios. This approach can simplify the interpretation of relative abundance by focusing on differences relative to a chosen reference.

Isometric log-ratio (ILR) transformation converts compositional data into a set of orthogonal coordinates using log-ratios. ILR transformation facilitates multivariate analysis and enhances the interpretability of complex compositional data.

Multi-additive log-ratio (MALR) transformation extends the additive log-ratio approach by incorporating multiple reference components or factors. This method allows for a more nuanced analysis of compositional data by addressing multiple dimensions of variability.

The microbiome sample may be evaluated using various methods. Nonlimiting examples include culture-based methods, molecular techniques, and quantitative PCR (qPCR). Historically, culture-based methods were the primary approach for studying bacterial populations. This involves isolating bacteria from skin samples and growing them on selective media. The amount of each species is inferred from colony counts or the presence of specific growth characteristics. This allows for the characterization of viable, culturable bacteria.

Molecular techniques do not require culturing the bacteria and include, for example, 16S rRNA gene sequencing and metagenomic sequencing. For 16S rRNA gene sequencing, microbiome samples are collected, for example, using swabs or tape strips. DNA is extracted from the collected samples and the 16S rRNA gene is amplified using polymerase chain reaction (PCR) with primers targeting conserved regions flanking hypervariable regions. Nonlimiting examples of useful primers include those that target the variable regions V1-V3 and V4. The amplified gene fragments are then sequenced using high-throughput sequencing technologies and analyzed to identify bacterial taxa and their amount. 16S rRNA gene sequencing is preferable because it provides comprehensive bacterial community profiles and does not require culturing, allowing for the detection of non-culturable species.

Metagenomic sequencing involves sequencing all genetic material in a sample, not just the 16S rRNA gene. This approach allows for the identification of bacteria as well as other microorganisms, and provides insights into functional potential. Samples are collected similar to 16S rRNA sequencing. The DNA in the sample is fragmented and sequencing libraries are prepared. The sequence data is analyzed to identify bacterial species and functional genes. This methods provides a broader view of the microbial community, including non-bacterial organisms.

Quantitative PCR (qPCR) is used to quantify the abundance of specific bacterial taxa or genes of interest. This technique measures the amount of DNA or RNA in real-time during the amplification process. Samples are collected similar to 16S rRNA sequencing and specific primers and probes are used to quantify the amount of target DNA. The amount of DNA is correlated with the abundance of the various bacterial species. This method provides quantitative data on specific bacterial groups and is relatively fast and less expensive than sequencing methods.

The control is typically the amount of bacteria in a microbiome sample from an individual or a population of individuals that are not suffering from acne or are not prone to developing acne. Comparing the amount of the bacteria in the microbiome sample from the skin of an individual prone to developing acne with a control allows for detection of difference in amounts between the microbiome sample from the skin of the individual prone to developing acne and the control. For example, an excess of abundance of one or more of the bacteria or an absence of abundance of one or more of the bacteria indicates that the individual is prone to developing acne. Difference indicate that the individual is prone to developing acne.

Controls can be developed with microbiome samples obtained from an individual or population of individuals not prone to developing acne or not currently suffering from acne. The lack of acne and historical resistance to developing acne suggests a balanced microbiome indicative of healthy skin. Microbiome samples from the skin of the individual or population of individuals not prone to developing acne or not currently suffering from acne can be assessed pursuant to the methods discussed above, e.g., with culture-based methods, molecular techniques, and quantitative PCR (qPCR). Such information can be used to develop a reference database of microbial profiles from healthy skin samples. Normative ranges are defined for the abundances of various bacterial taxa for use as a control profile. Using this control profile as a benchmark, the microbiome of skin from individuals can be evaluated relative to the benchmark. Differences between the microbiome and the benchmark are identified and can be monitored to assess changes to the skin's microbiota in response to interventions, i.e., in response to treatment. Developing a control for a healthy skin microbiome involves a systematic approach that includes defining health criteria, recruiting a representative sample population, collecting and processing samples, performing detailed microbiome analysis, and creating a reference profile.

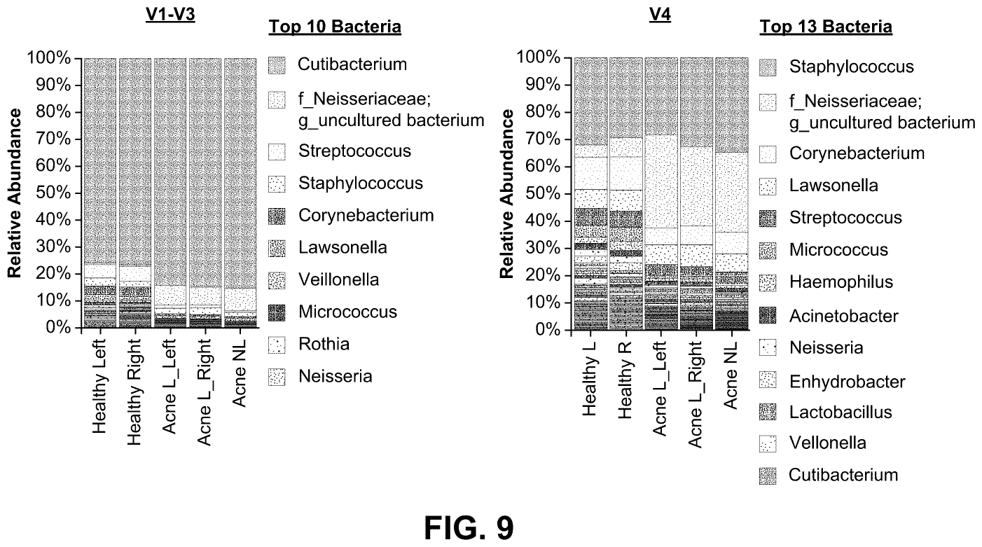

A nonlimiting example of a reference profile is shown in FIG. 9. FIG. 9 shows bacterial profile from skin samples collected from Right (R) and Left (L) cheeks of healthy individuals, which may be considered a control. The microbiome samples were analyzed using 16S ribosomal RNA (16S rRNA) amplicon sequencing with primers that targeted the variable regions (V1-V3) and V4.

An individual identified as an individual prone to develop acne may be treated to prevent development of acne. The treatment involves minimizing or eliminating differences between the abundance of bacteria in the microbiome of the individual's skin and the abundance of the bacteria in the control. If the abundance of a particular bacteria in the microbiome of the individual's skin is higher than the control for a particular bacteria, the individual is treated to reduce the abundance of the particular bacteria. If the amount of a particular bacteria is lower than the control, the individual is treated to increase the abundance of the particular bacteria.

Upon identification of an individual prone to developing acne, the individual can be treated to prevent the individual from developing acne or to reduce the likelihood the individual will develop acne. Typically, the individual is treated to restore the amounts of the bacteria of the individual's skin to a normal and healthy equilibrium. This involves minimizing differences between the amount of the bacteria on the skin of the individual and the control. Nonlimiting examples of treatment that can be employed to minimize differences between the microbiome of the individual's skin and the control include topical treatments, oral treatments, physical treatments, and energy-based treatments. The treatments serve to align the amounts of bacteria in the microbiome of the individual's skin with the control.

In a preferred embodiment, the treatment comprises topically administering to the individual's skin a pharmaceutical or cosmetic composition that minimizes the difference between the amount of the bacterial in the microbiome of the individual's skin and the control. The pharmaceutical or cosmetic composition typically includes a therapeutically effective amount of one or more anti-acne compounds. Nonlimiting examples of anti-acne compounds include retinoids, anti-bacterial compounds, anti-inflammatory compounds, essential oils, botanical extracts, probiotics, prebiotics, postbiotics, metabolites, antibiotics, sulfur, vitamins, and benzoyl peroxide.

Nonlimiting examples of retinoids include tretinoin, adapalene, tazarotene, retinol, and retinaldehyde). Nonlimiting examples of anti-bacterial compounds include benzoyl peroxide, salicylic acid, tea tree oil, triclosan, clindamycin, azelaic acid, sulfur, hydrogen peroxide, chlorhexidine, iodine-based compounds, lactic acid, hyaluronic acid, and antimicrobial peptides. Nonlimiting examples of anti-inflammatory compounds include niacinamide, salicylic acid, benzoyl peroxide, azelaic acid, sulfur, green tea extract, chamomile extract, aloe vera, licorice extract, calendula extract, resveratrol, bisabolol, allantoin, honey, propolis, turmeric extract, centella asiatica, sea buckthorn oil, tea tree oil, witch hazel, rooibos extract, oat extract, panthenol, arnica, hemp seed oil, ginger extract, neem oil, grapeseed oil, evening primrose oil, meadowfoam seed oil, cica (Centella Asiatica), purslane extract, vitamin E, vitamin C, rosemary extract, and alpha-lipoic acid. Nonlimiting examples of essential oils include tea tree oil, lavender oil, peppermint oil, eucalyptus oil, rosemary oil, chamomile oil, geranium oil, clary sage oil, frankincense oil, and Ylang-Ylang oil). In various embodiments, the pharmaceutical or cosmetic composition includes, as anti-acne compounds, one or more botanical extracts. Nonlimiting examples of botanical extracts include chamomile extract, green tea extract, witch hazel extract, aloe vera extract, calendula extract, licorice root extract, turmeric extract, neem extract, burdock root extract, rosehip oil, Gotu Kola extract, and sage extract).

The pharmaceutical or cosmetic composition may also include one or more probiotics and/or one or more prebiotics for use as anti-acne compounds. Nonlimiting examples of probiotics include Lactobacillus acidophilus, Lactobacillus rhamnosus, Bifidobacterium bifidum, Lactoplantibacillus plantarum, Bifidobacterium longum, Streptococcus thermophilus, and Lactobacillus casei. Nonlimiting examples of prebiotics include inulin, fructooligosaccharides, alpha-glucan oligosaccharides, glycosaminoglycans, Lactobacillus ferment lysate, Bifidobacterium ferment lysate, Saccharomyces ferment filtrate, oligosaccharides from a plant source, and probiotic lysates). Vitamins are also useful anti-acne compounds. Nonlimiting examples include vitamin A, vitamin C, vitamin E, vitamin B3 (niacinamide), vitamin B5, vitamin D, and vitamin K.

The pharmaceutical or cosmetic composition may be applied to the skin once daily, twice daily, once weekly, twice weekly, thrice weekly, every-other-day, once monthly, twice monthly, or more than twice monthly.

In a preferred embodiment, the treatment comprises orally administering to the individual a therapeutically effective amount of an active compound that minimizes the difference between the amount of the bacterial in the microbiome of the individual's skin and the control. Nonlimiting examples of active compounds for oral administration include antibiotics, hormones, zinc sources (e.g., zinc salts), prebiotics, postbiotics, metabolites, probiotics, omega-3-fatty acids, and vitamins.

Nonlimiting examples of antibiotics include doxycycline, minocycline, tetracycline, clindamycin, erythromycin, azithromycin, amoxicillin, cephalexin, ciprofloxacin, levofloxacin, moxifloxacin, trimethoprim-sulfamethoxazole, rifampin, metronidazole, ampicillin, linezolid, vancomycin, chloramphenicol, norfloxacin, ofloxacin, doxycycline hyclate, penicillin, cefuroxime, ceftriaxone, cefotaxime, ampicillin-sulbactam, sulfisoxazole, minocycline hydrochloride, tetracycline hydrochloride, tigecycline, ertapenem, imipenem-cilastatin, meropenem, and doripenem.

Nonlimiting examples of hormones include Estrogen, progesterone, testosterone, prednisone, hydrocortisone, dexamethasone, medroxyprogesterone acetate, norethindrone, estradiol, levonorgestrel, desogestrel, drospirenone, estrone, estriol, cyproterone acetate, and finasteride.

Nonlimiting examples of zinc salts include zinc sulfate, zinc gluconate, zinc acetate, zinc citrate, zinc oxide, zinc picolinate, zinc aspartate, and zinc monomethionine.

Nonlimiting examples of prebiotics include inulin, fructooligosaccharides (FOS), galactooligosaccharides (GOS), lactulose, xylooligosaccharides (XOS), oligosaccharides, beta-glucans, psyllium husk, resistant starch, prebiotic fibers derived from acacia gum, arabinogalactan, maltodextrin (with prebiotic effects), glucomannan, isomaltooligosaccharides (IMO), oligofructose, polydextrose, mannanoligosaccharides (MOS), and pectin.

Nonlimiting examples of probiotics include Lactobacillus acidophilus, Lactobacillus rhamnosus, Lactobacillus reuteri, Lactobacillus casei, Lactoplantibacillus plantarum, Lactobacillus fermentum, Lactobacillus helveticus, Lactobacillus salivarius, Lactobacillus gasseri, Lactobacillus johnsonii, Lactobacillus paracasei, Lactobacillus delbrueckii, Lactobacillus bulgaricus, Lactobacillus brevis, Lactobacillus lactis, Bifidobacterium bifidum, Bifidobacterium longum, Bifidobacterium infantis, Bifidobacterium breve, Bifidobacterium lactis, Bifidobacterium adolescentis, Bifidobacterium pseudocatenulatum, Bifidobacterium animalis, Bifidobacterium thermophilum, Streptococcus thermophilus, Saccharomyces boulardii, Saccharomyces cerevisiae, Enterococcus faecium, Enterococcus faecalis, and Propionibacterium freudenreichii.

Nonlimiting examples of omega-3-fatty acids include alpha-linolenic acid (ALA), eicosapentaenoic acid (EPA), docosahexaenoic acid (DHA), stearidonic acid (SDA), eicosatetraenoic acid (ETA), docosapentaenoic acid (DPA), linolenic acid, 6,9,12-octadecatrienoic acid (also known as ALA), and 8,11,14-eicosatrienoic acid (also known as ETA).

Nonlimiting examples of vitamins include Vitamin A, Vitamin B1 (thiamine), Vitamin B2 (riboflavin), Vitamin B3 (niacin), Vitamin B5 (pantothenic acid), Vitamin B6 (pyridoxine), Vitamin B7 (biotin), Vitamin B9 (folate or folic acid), Vitamin B12 (cobalamin), Vitamin C (ascorbic acid), Vitamin D, Vitamin E, Vitamin K1 (phylloquinone), Vitamin K2 (menaquinone), Vitamin D2 (ergocalciferol), Vitamin D3 (cholecalciferol), Vitamin A1 (retinol), Vitamin A2 (dehydroretinol), and Vitamin K3 (menadione).

The active compounds may be administered to the individual once daily, twice daily, once weekly, twice weekly, thrice weekly, every-other-day, once monthly, twice monthly, or more than twice monthly.

In a preferred embodiment, the treatment comprises administering a therapeutically effective amount of a light therapy (or energy therapy) to the individual. Nonlimiting examples of light therapy include blue light therapy, red light therapy, yellow light therapy, green light therapy, intense pulsed light therapy, photodynamic therapy, UV light therapy, and infrared therapy. The light therapy can be administered once daily, twice daily,

The light therapy may be applied to the skin once daily, twice daily, once weekly, twice weekly, thrice weekly, every-other-day, once monthly, twice monthly, thrice monthly, every three months, every six month, or as needed.

In various embodiments, the method includes further testing the microbiome of the individual after treatment and assessing the amount of the bacteria to determine how the amount of the bacteria has changed in response to treatment. For instance, a microbiome sample is obtained from the skin of the individual who has undergone treatment and the amount of the bacteria in the microbiome sample determined, for example, according to the methods outlined above. In particular, the amount of Lawsonella, Cutibacterium, Staphylococcus, Corynebacterium, Streptococcus, Micrococcus, and/or Neisseriaceae is determined. The amount of the bacteria can be compared with the amount of the bacteria present in the initial microbiome sample taken before treating the individual. Changes in the amount are identified. Similarly, the amount of the bacteria can be compared with the control and differences identified. Based on the changes in the amount of the bacterial after treatment, the treatment can be updated as needed.

The treatment involves minimizing or eliminating differences between the abundance of bacteria in the microbiome of the individual's skin and the abundance of the bacteria in the control. If the abundance of a particular bacteria in the microbiome of the individual's skin is higher than the control for a particular bacteria, the individual is treated to reduce the abundance of the particular bacteria. If the amount of a particular bacteria is lower than the control, the individual is treated to increase the abundance of the particular bacteria.

Methods for determining severity of acne lesions for an individual suffering from acne lesions are also important. Such methods allow for identification of underlying causes of the acne lesions and provide guidance for treating and prevent future occurrence of acne lesions. Accordingly, the instant disclosure is drawn to a method for determining severity of acne lesions for an individual suffering from acne lesions, the method comprising:
(a) obtaining microbiome sample from skin of the individual suffering from acne lesions;
(b) determining an amount of one or more bacteria in the microbiome sample, wherein the one or more bacteria are selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof;
(c) comparing the amount of the bacteria to a control;
(d) identifying how the amount of the one or more bacteria in the microbiome sample differs from the control with respect to abundance of at least one bacteria selected from Lawsonella, Cutibacterium, Staphylococcus, Corynebacterium, Streptococcus, Micrococcus, and Neisseriaceae, preferably wherein the at least one bacteria include Lawsonella, Cutibacterium, or a combination thereof; and
(e) and determining the severity of acne lesions based on the difference.

The microbiome sample is obtained from the skin of an individual suffering from acne lesions. The microbiome samples may be collected from lesional and/or non-lesional areas of skin. For example, the microbiome sample is typically from skin of the face, skin of the back, and skin of the shoulders The microbiome sample is evaluated to determine the amount of the bacterial in the microbiome sample. Preferably, the amount of more than one bacteria is evaluated. For example, the amount of two or more, three or more, four or more, five or more, six or more, or all bacteria are evaluated, wherein the bacteria are selected from Lawsonella, Cutibacterium, Staphylococcus, Corynebacterium, Streptococcus, Micrococcus, and Neisseriaceae. The amount of the bacteria in the microbiome sample can be determined using the methods discussed above, i.e., culture-based methods, molecular techniques, and quantitative PCR (qPCR).

Determining an amount of one or more bacteria in the microbiome sample involves quantifying the amount of the bacteria. The quantification of bacteria can be expressed in several ways, including absolute abundance, relative abundance, and through various log-ratio transformations. Absolute abundance refers to the total number of bacterial cells present in the sample, offering a direct count without comparison to other components. This measure provides a straightforward assessment of bacterial load. Relative abundance, on the other hand, calculates the proportion of each bacterial species relative to the total bacterial community in the sample. This approach is useful for understanding the composition of bacterial communities and the relative contributions of different species to the overall microbial landscape.

Preferably, log-ratio transformations are employed. Examples of log-ratio transformations include the robust centered log-ratio (RCLR) transformation, centered log-ratio (CLR) transformation, additive log-ratio (ALR) transformation, isometric log-ratio (ILR) transformation, and multi-additive log-ratio (MALR) transformation. Among these, the robust centered log-ratio (RCLR) transformation is preferred.

Controls can be developed using microbiome samples obtained from an individual or population of individuals not suffering from acne lesions and/or not historically prone to developing acne. The lack of acne lesions and/or historical resistance to developing acne suggests a balanced microbiome indicative of healthy skin. Microbiome samples from the skin of the individual or population of individuals not currently suffering from acne lesions and/or not prone to developing acne can be evaluating with the methods discussed above, e.g., with culture-based methods, molecular techniques, and quantitative PCR (qPCR). The information is used to develop a reference database of microbial profiles from healthy skin samples. Normative ranges are defined for the amounts of various bacterial taxa for use as a control profile. Using this control profile as a benchmark, the microbiome of skin from individuals can be evaluated relative to the benchmark. Differences between the microbiome and the benchmark are identified and can be monitored to assess changes to the skin's microbiota in response to interventions, i.e., in response to treatment. Developing a control for a healthy skin microbiome involves a systematic approach that includes defining health criteria, recruiting a representative sample population, collecting and processing samples, performing detailed microbiome analysis, and creating a reference profile.

The individual suffering from acne lesions can be treated to prevent continued occurrence of acne lesion or to reduce their severity. The treatment involves minimizing or eliminating differences between the abundance of bacteria in the microbiome of the individual's skin and the abundance of the bacteria in the control. If the abundance of a particular bacteria in the microbiome of the individual's skin is higher than the control for a particular bacteria, the individual is treated to reduce the abundance of the particular bacteria. If the abundance of a particular bacteria is lower than the control, the individual is treated to increase the abundance of the particular bacteria.

Upon identification of the differences between the microbiota of the individual suffering from acne lesions and the control, the individual can be treated to minimize the differences. Typically, the individual is treated to restore the amount of the bacteria of the individual's skin to a normal and healthy equilibrium according to the control. Nonlimiting examples of treatment that can be employed to minimize differences between the microbiome of the individual's skin and the control are discussed above, and include topical treatments, oral treatments, physical treatments, and energy-based treatments. Nonlimiting examples of the various treatments are discussed above and incorporated herein by reference. The treatments serve to align the amount of bacteria in the microbiome of the individual's skin with the control.

In various embodiments, the method includes further testing the microbiome of the individual after treatment and assessing the amount of the bacteria to determine how the amount of the bacteria has changed in response to treatment. For instance, a microbiome sample is obtained from the skin of the individual who has undergone treatment and the amount of the bacteria in the microbiome sample determined, for example, according to the methods outlined above. In particular, the amount of Lawsonella, Cutibacterium, Staphylococcus, Corynebacterium, Streptococcus, Micrococcus, and/or Neisseriaceae is determined. The amount of the bacteria can be compared with the amount of the bacteria present in the initial microbiome sample taken before treating the individual. Changes in the amount are identified. Similarly, the amount of the bacteria can be compared with the control and differences identified. Based on the changes in the amount of the bacterial after treatment, the treatment can be updated as needed.

The methods of the instant disclosure are particularly useful for personalized prevention and optional treatment of acne. The microbiome of the individual's skin is assessed and the unique differences between the individual microbiome and a control identified. Treatment is tailed to the specific needs of the individual as elucidated by the unique differences between the individual's microbiome and the control. For example, a method for personalized prevention and treatment of acne comprising:
(a) obtaining microbiome sample from skin of an individual;
(b) determining an amount of one or more bacteria in the microbiome sample, wherein the one or more bacteria are selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof;
(c) comparing the amount of the bacteria to a control;
(d) identifying a difference between the amount of the one or more bacteria in the microbiome sample and the control with respect to at least one bacteria selected from Lawsonella, Cutibacterium, Staphylococcus, Corynebacterium, Streptococcus, Micrococcus, and Neisseriaceae, preferably wherein the one at least one bacteria include Lawsonella, Cutibacterium, or a combination thereof; and
(c) treating the individual by minimizing the difference between the amount of the bacteria in the microbiome of the individual's skin and the control.

The microbiome sample is from skin that is prone to develop acne, for example, skin of the face, skin of the back, and skin of the shoulders.

The microbiome sample is evaluated to determine the amount of the bacterial in the microbiome sample. In particular, the microbiome sample is evaluated to determine the amount of one or more bacteria selected from Lawsonella, Cutibacterium, Staphylococcus, Corynebacterium, Streptococcus, Micrococcus, and Neisseriaceae. In many cases, the amount of more than one bacteria is evaluated. For example, the amount of two or more, three or more, four or more, five or more, six or more, or all bacteria are evaluated, wherein the bacteria are selected from Lawsonella, Cutibacterium, Staphylococcus, Corynebacterium, Streptococcus, and Micrococcus, and Neisseriaceae. The amount of the bacteria in the microbiome sample can be determined using the methods discussed above, i.e., culture-based methods, molecular techniques, and quantitative PCR (qPCR).

Determining an amount of one or more bacteria in the microbiome sample involves quantifying the amount of the bacteria. The quantification of bacteria can be expressed in several ways, including absolute abundance, relative abundance, and through various log-ratio transformations. Absolute abundance refers to the total number of bacterial cells present in the sample, offering a direct count without comparison to other components. This measure provides a straightforward assessment of bacterial load. Relative abundance, on the other hand, calculates the proportion of each bacterial species relative to the total bacterial community in the sample. This approach is useful for understanding the composition of bacterial communities and the relative contributions of different species to the overall microbial landscape.

Preferably, log-ratio transformations are employed. Examples of log-ratio transformations include the robust centered log-ratio (RCLR) transformation, centered log-ratio (CLR) transformation, additive log-ratio (ALR) transformation, isometric log-ratio (ILR) transformation, and multi-additive log-ratio (MALR) transformation. Among these, the robust centered log-ratio (RCLR) transformation is preferred.

A control can be developed with microbiome samples obtained from an individual or population of individuals not prone to developing acne or not currently suffering from acne. The lack of acne and historical resistance to developing acne suggests a balanced microbiome indicative of healthy skin. Microbiome samples from the skin of the individual or population of individuals not prone to developing acne or not suffering from acne can be assessed pursuant to the methods discussed above, e.g., with culture-based methods, molecular techniques, and quantitative PCR (qPCR). Such information can be used to develop a reference database of microbial profiles from healthy skin samples. Normative ranges are defined for the amounts of various bacterial taxa for use as a control profile. Using this control profile as a benchmark, the microbiome of skin from individuals can be evaluated relative to the benchmark. Differences between the microbiome and the benchmark are identified and can be monitored to assess changes to the skin's microbiota in response to interventions, i.e., in response to treatment. Developing a control for a healthy skin microbiome involves a systematic approach that includes defining health criteria, recruiting a representative sample population, collecting and processing samples, performing detailed microbiome analysis, and creating a reference profile.

Based on the differences in amounts of bacteria between the microbiome sample and the control, a personalized treatment can be implemented to minimize differences between the abundance of the bacteria on the individual skin and the control. Typically, the individual is treated to restore the amount of the bacteria of the individual's skin to a normal and healthy equilibrium. Nonlimiting examples of treatment that can be employed to minimize differences between the microbiome of the individual's skin and the control include topical treatments, oral treatments, physical treatments, and energy-based treatments. Nonlimiting examples of the various treatments are discussed above and incorporated herein by reference.

In various embodiments, the method includes further testing the microbiome of the individual after treatment and assessing the amount of the bacteria to determine how the amount of the bacteria has changed in response to treatment. For instance, a microbiome sample is obtained from the skin of the individual who has undergone treatment and the amount of the bacteria in the microbiome sample determined, for example, according to the methods outlined above. In particular, the amount of Lawsonella, Cutibacterium, Staphylococcus, Corynebacterium, Streptococcus, Micrococcus, and/or Neisseriaceae is determined. The amount of the bacteria can be compared with the amount of the bacteria present in the initial microbiome sample taken before treating the individual. Changes in the amount are identified. Similarly, the amount of the bacteria can be compared with the control and differences identified. Based on the changes in the amount of the bacterial after treatment, the treatment can be updated as needed.

Various changes can be made in the above-described methods without departing from the scope of the invention. Accordingly, it is intended that all disclosure contained in the above description and in the examples given below, shall be interpreted as illustrative and not in a limiting sense.

### EXAMPLES

### Example 1

### Longitudinal Clinical Study

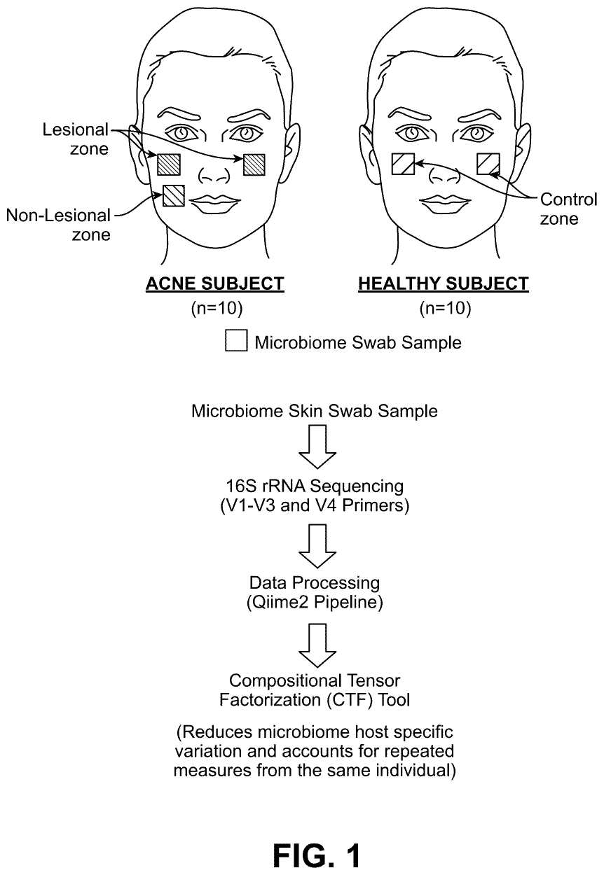

A longitudinal clinical study was carried out to identify microbiome markers associated with acne and acne lesion evaluation. Ten individuals suffering from acne (acne subjects) and ten individuals not suffering from acne (healthy subjects) participated in the study over a period of twenty-eight days. Skin microbiome samples were collected every-other-day using swabs from lesion areas (n=142 samples) and from non-lesion areas (n=47 samples) from the faces of the individuals suffering from acne. In addition, samples were collected from non-lesion areas (control zones) from the faces of the healthy individuals (n=47 samples) (control). FIG. 1 illustrates the testing procedure.

The microbiome samples were analyzed using 16S ribosomal RNA (16S rRNA) amplicon sequencing with primers that targeted the variable regions (V1-V3) and V4. 16S rRNA is an integral part of the 30S ribosomal subunit in bacteria and plays a vital role in the process of protein synthesis by facilitating the translation of messenger RNA (mRNA) into proteins. It contributes to the structural framework of the ribosome, helping to maintain its stability and function. The sequence of the 16S rRNA gene is highly conserved, making it a valuable tool for identifying and classifying bacteria. The gene sequence varies enough between different genera and some species to allow for the differentiation of bacterial taxa. Polymerase chain reaction (PCR) and sequencing are used to identify bacterial taxa and study microbial diversity.

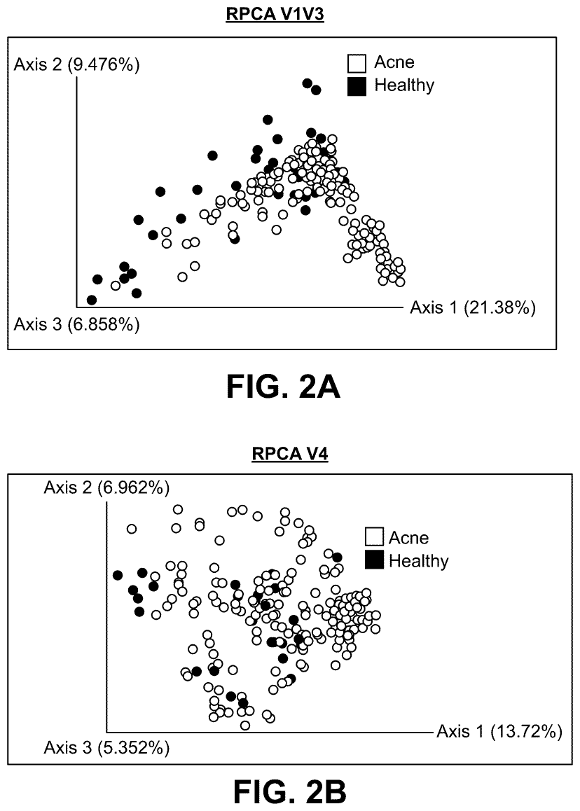

The 16S rRNA gene has both conserved and variable regions. The conserved regions are similar across different bacterial species, while the variable regions (V1, V2, V3, V4, etc.) differ between species and can provide information about bacterial diversity and taxonomy. V1-V3 primers target the V1 to V3 regions of the 16S rRNA gene. Amplifying these regions provides a broad overview of bacterial diversity. V4 primers target the V4 region of the 16S rRNA gene. The V4 region is used because it provides a good balance between sequence length and taxonomic resolution. By using these primers to amplify variable regions of bacteria and then sequencing them, different bacterial species present in a sample are identified and quantified. Notwithstanding high inter-individual variability and host microbiome variations, separation was observed between samples from acne subjects and healthy subjects with Robust Aitchison Principal Components Analysis (RPCA) beta diversity analysis, as shown in FIG. 2(a) (V1-V3 primers) and FIG. 2(b) (V4 primers).

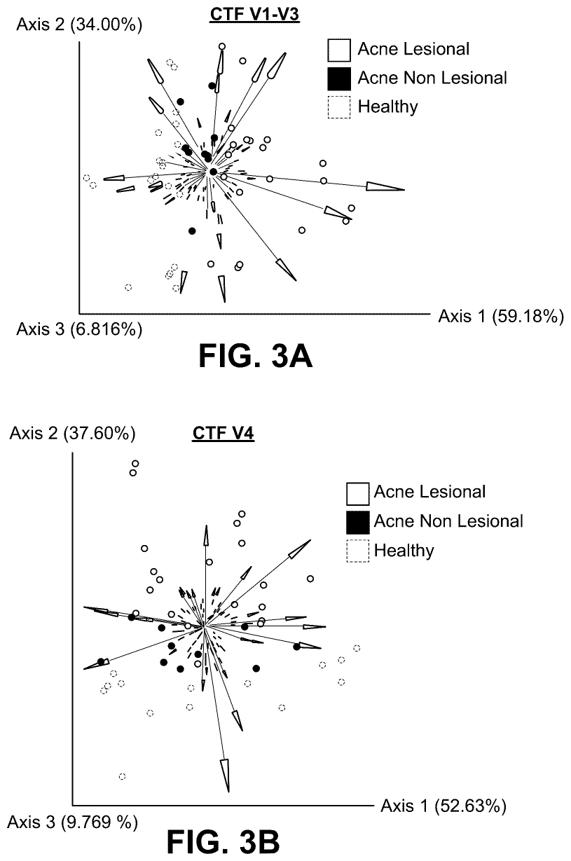

To more accurately pinpoint unique markers associated with acne while accounting for host microbiome variation and repeated measures from the same individual, the Center for Microbiome Innovation at the University of California, San Diego (CMI-UCSD) further evaluated the data using an advanced dimensionality reduction analysis Compositional Tensor Factorization (CTF). CTF is an advanced mathematical technique used to decompose and analyze multi-dimensional data structures, known as tensors. Tensors are generalizations of matrices to higher dimensions and can represent complex data such as multi-way relationships or multi-modal information, which is described by Martino et al., Compositional Tensor Factorization for Robust and Interpretable Analysis of High-Dimensional Data, IEEE TRANSACTIONS ON KNOWLEDGE AND DATA ENGINEERING, 33(5):2121-2132 (2021); DOI: 10.1109/TKDE.2020.3019637. CTF analysis revealed a clear separation between microbiome profiles of samples from the acne subject and healthy subjects, as shown in FIG. 3(a) (V1-V3 primers) and FIG. 3(b) (V4 primers).

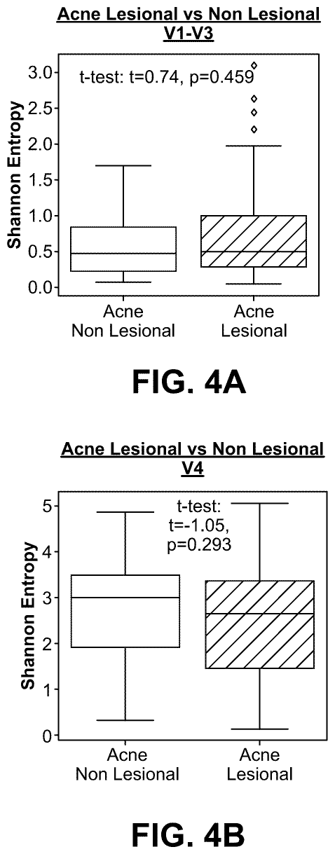

The analysis also included microbiome profiles of samples from non-lesional areas of acne subjects, as an intermediate profile between the lesional areas of the acne subjects and the healthy subjects. The data (FIG. 3(a) and FIG. 3(b)) suggests possible evolution between lesion status and healthy status. The differentiation of profile occurs in the absence of significant differences in bacterial richness (alpha diversity) between lesional and non-lesional samples collected from acne subjects at baseline and over 28 days, as shown in FIG. 4. Similar patterns are observed for 16S rRNA data sequenced using V1-V3 (FIG. 2(a), FIG. 3(a), and FIG. 4) and V4 (FIG. 2(b), FIG. 3(b), and FIG. 4).

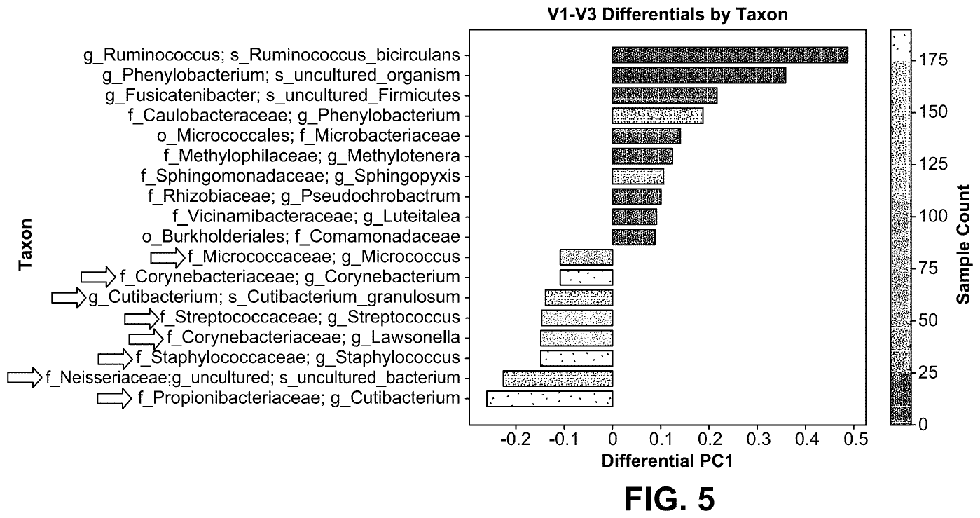

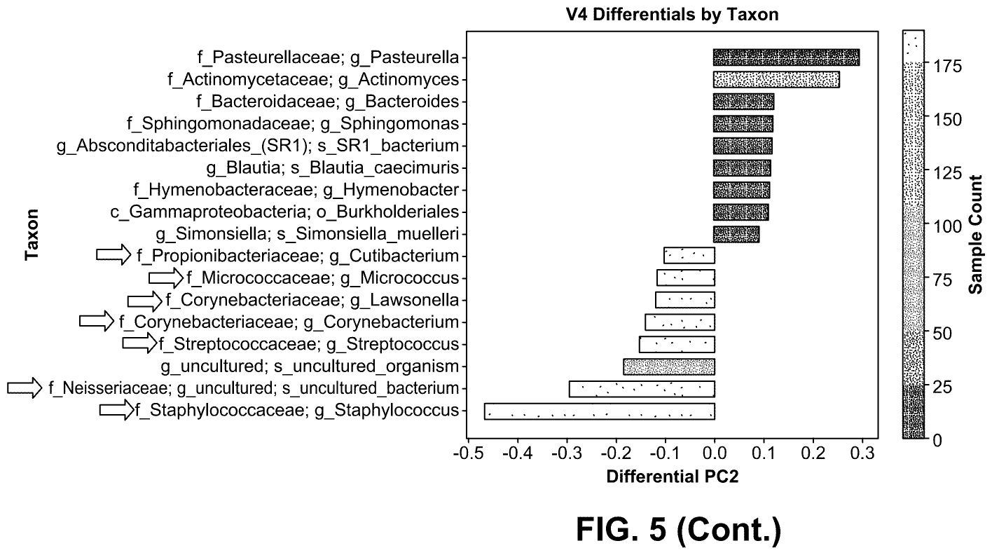

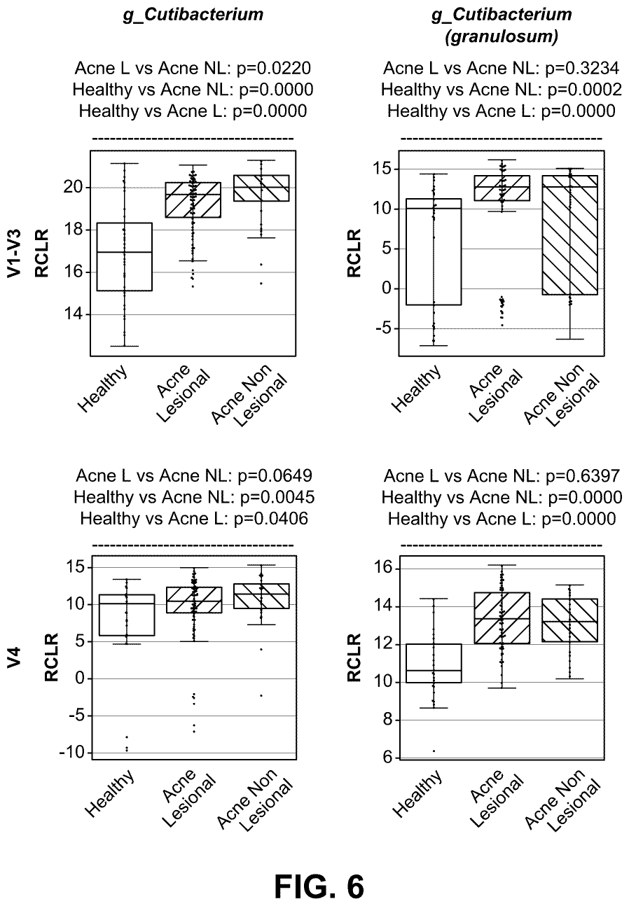

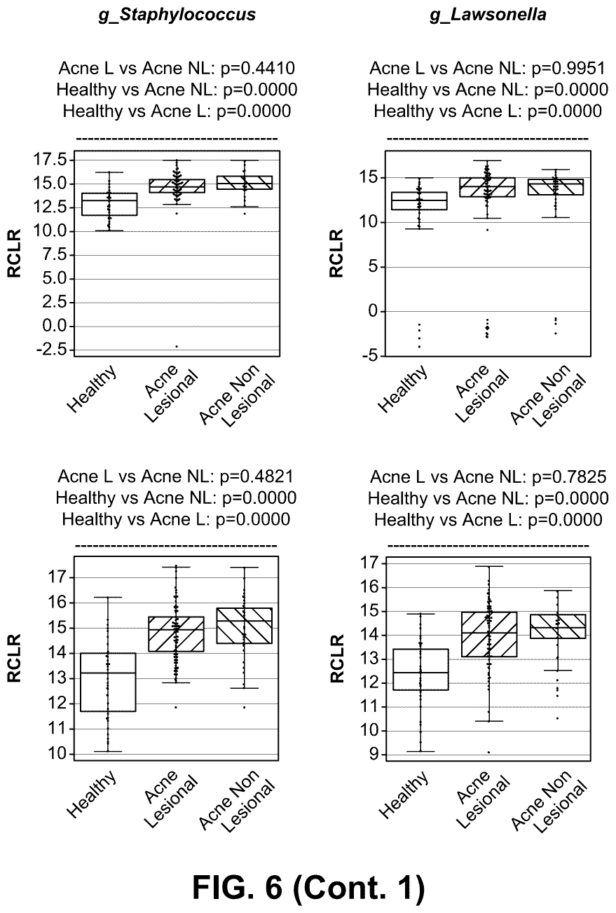

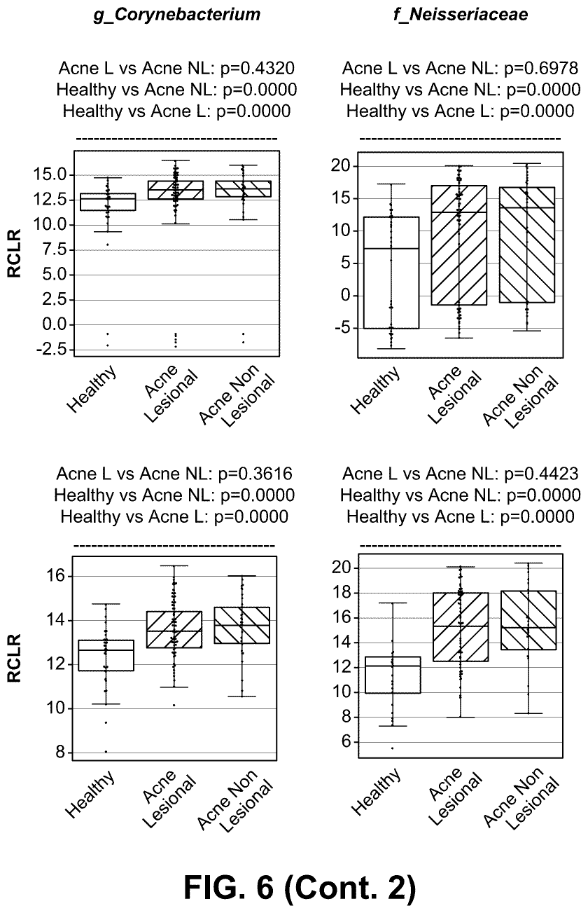

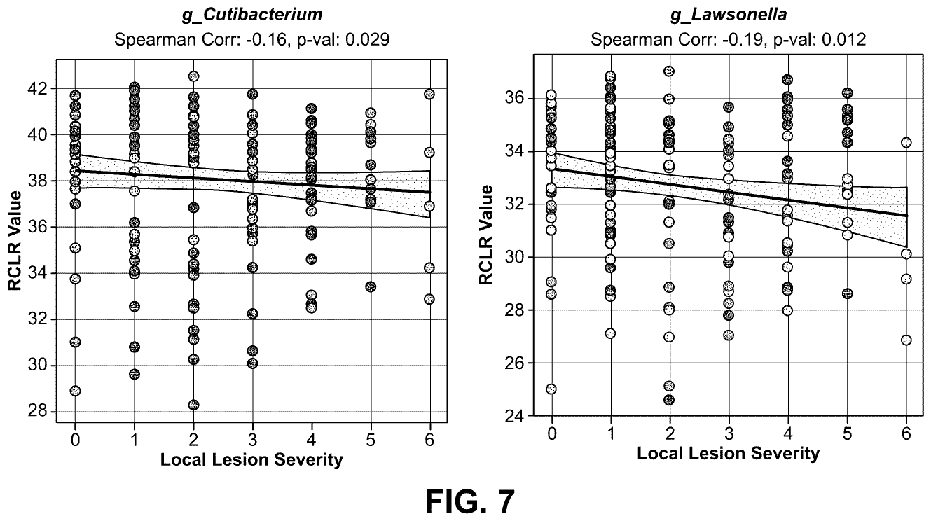

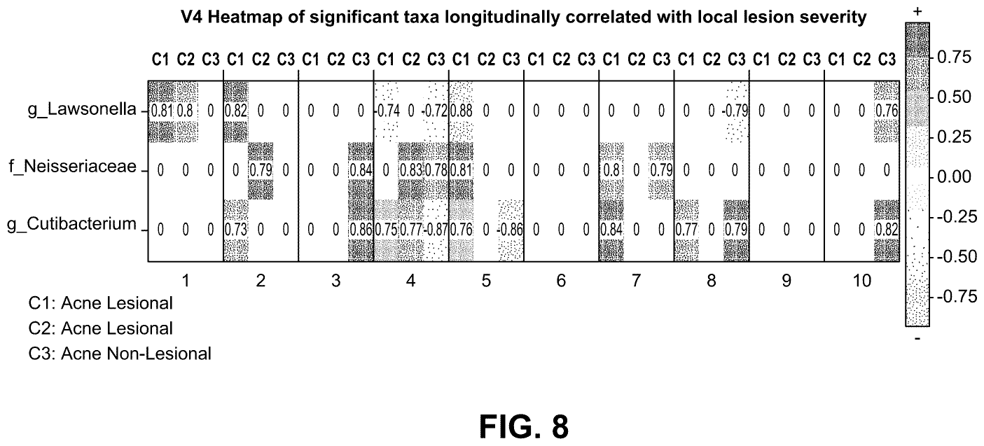

Key microbial markers that drive the separation between acne and healthy samples were identified, including: Lawsonella, Cutibacterium, Staphylococcus, Corynebacterium, Streptococcus, Micrococcus, and Neisseriaceae, as shown in FIG. 5. The robustness of the findings is highlighted by the fact that the same markers were identified from the V1-V3 and V4 data. The abundance of Lawsonella, Cutibacterium, Staphylococcus, Corynebacterium, Neisseriaceae, represented as RCLR transformations, was significantly higher in both lesional and non-lesional acne samples compared to samples from healthy subjects, as shown in FIG. 6. Acne Markers Lawsonella and Cutibacterium also display an inverse correlation with local lesion severity globally, i.e., their abundance decreases with increasing local lesion severity (FIG. 7), possibly due to species-specific responses. Additionally, we identified bacterial features which also tend to track, either in the same direction or opposite direction, with lesion severity over time, including ASVs classified as belonging to Cutibacterium, Lawsonella, Neisseriaceae, correlating with lesion progression in 7/10, 6/10 and 5/10 subjects, respectively, as shown by FIG. 8.

FIG. 9 shows bacterial profiles (relative abundance of taxa) from skin samples collected from Right (R) and Left (L) cheeks of healthy individuals, which may be considered a control. The microbiome samples were analyzed using 16S ribosomal RNA (16S rRNA) amplicon sequencing with primers that targeted the variable regions (V1-V3) and V4.

The foregoing description illustrates and describes preferred embodiments of the inventions. Nonetheless, it is to be understood that the inventions are capable of use in various other combinations, modifications, and environments and are capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the inventions to the forms disclosed herein. Also, it is intended to the appended claims be construed consistent with the specification and to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art. The disclosure may not expressly identify all compounds mentioned throughout the disclosure that may exist as a salt or may exist in an ionized form, for example, when formulated in a pharmaceutical or cosmetic composition. Nonetheless, to the extent salt or ionized forms of the compound exist and are known, they are intended to be encompassed within the scope of the instant disclosure, even if not expressly mentioned. For example, many surfactants can exist as salts or be in an ionized form in pharmaceutical or cosmetic compositions. Likewise, peptides may be in the form of salts or ionized, which can improve their stability or compatibility. Nonlimiting examples of useful anions include sodium, potassium, calcium, magnesium, chloride, sulfate, phosphate, acetate, citrate, TFA (trifluoroacetate), bicarbonate, and nitrate.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions may be modified in all instances by the term “about,” meaning within +/−5% of the indicated number. For example, an amount of “about 10 wt. %” includes amounts as low as 9.5 wt. % and as high as 10.5 wt. %. An amount of “about 50 wt. %” includes amounts as low as 47.5 wt. % and as high as 52.5 wt. %.

“Abundance” refers to the quantity or amount of something. In a broad sense, it can apply to any countable or measurable item, whether it is organisms in an ecosystem, resources, or anything else that can be quantified. The term “relative abundance,” on the other hand, specifically refers to the proportion or percentage of a particular item or species in relation to the total number of items or species in a given area or sample. It provides context by comparing the quantity of one item to the quantities of others within the same system.

All percentages, parts and ratios herein are based upon weight, unless otherwise indicated.

Some of the various categories of components mentioned throughout the disclosure may overlap. Nonetheless, a single overlapping component cannot simultaneously serve as two different components despite the overlap. Certain alcohols function as both a preservative and as a water-soluble solvent, which may be useful in a physiologically acceptable carrier. If an embodiment, claim, or other recitation throughout the disclosure communicates that both a preservative and a water-soluble solvent are present, needed, or required, a single alcohol such as ethanol cannot simultaneously serve as the preservative and as the water-soluble solvent. In this case, the ethanol shall serve as the preservative or as the water-soluble solvent, but simultaneously as both.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein up to 2 wt. % of the element may be present. In further embodiments, however, up to 1.5 wt. %, up to 1 wt. %, up to 0.5 wt. %, up to 0.1 wt. %, up to 0.01 wt. % of the element may be present. For example, in the case of a pharmaceutical or cosmetic composition being substantially free or essentially free of a component, the pharmaceutical or cosmetic composition may include up to 2 wt. % of the component. Nonetheless, the pharmaceutical or cosmetic composition preferably includes 1.5 wt. % or less, 1 wt. % or less, 0.5 wt. % or less, 0.1 wt. % or less, 0.01 wt. % or less, or none of the specified component.

As used herein, a “subject” or an “individual” can be a human.

All components that are positively set forth throughout the disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A method for identifying an individual having a predisposition to developing acne, the method comprising:
(a) obtaining microbiome sample from skin of an individual;
(b) determining an amount of one or more bacteria in the microbiome sample, wherein the one or more bacteria are selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof;
(c) comparing the amount of the one or more bacteria to a control;
(d) identifying a difference between the amount of the one or more bacteria in the microbiome sample and the control with respect to at least one bacteria selected from Lawsonella, Cutibacterium, Staphylococcus, Corynebacterium, Streptococcus, Micrococcus, and Neisseriaceae, preferably wherein the at least one bacteria include Lawsonella, Cutibacterium, or a combination thereof; and
(c) identifying the individual having a predisposition to developing acne based on the difference.

2. The method of claim 1, wherein the control is an amount of bacteria in a microbiome sample from a population of individuals not suffering from acne, wherein the bacteria is at least one bacteria selected from Lawsonella, Cutibacterium, Staphylococcus, Corynebacterium, Streptococcus, Micrococcus, and Neisseriaceae, preferably wherein the at least one bacteria is Lawsonella, Cutibacterium, or a combination thereof.

3. The method of claim 1, further comprising, treating the individual having a predisposition to developing acne by minimizing the difference between amounts of the bacteria in the microbiome of the individual's skin and the control.

4. The method of claim 3, wherein the treatment comprises topically administering to the individual's skin a pharmaceutical or cosmetic composition that minimizes the difference between amounts of the bacterial in the microbiome of the individual's skin and the control.

5. The method of claim 4, wherein the pharmaceutical or cosmetic composition includes one or more anti-acne compounds selected from retinoids, anti-bacterial compounds, anti-inflammatory compounds, essential oils, a botanical extracts, probiotics, prebiotics, postbiotics, metabolites, vitamins, benzoyl peroxide, sulfur, or combinations thereof.

6. The method of claim 3, wherein the treatment comprising orally administering an active compound that minimizes the difference between the amounts of the bacterial in the microbiome of the individual's skin and the control.

7. The method of claim 6, wherein the compound is a retinoid, an antibiotic, a hormone, a zinc salt, a prebiotic, a probiotic, an omega-3-fatty acid, a vitamin, or combinations thereof.

8. A method for determining severity of acne lesions for an individual suffering from acne, the method comprising:
(a) obtaining microbiome sample from skin of the individual suffering from acne;
(b) determining an amount of one or more bacteria in the microbiome sample, wherein the bacteria are selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof;
(c) comparing the amount of the one or more bacteria to a control;
(d) identifying how the amount of the one or more bacteria in the microbiome sample differs from the control with respect to an amount of at least one bacteria selected from Lawsonella, Cutibacterium, Staphylococcus, Corynebacterium, Streptococcus, Micrococcus, and Neisseriaceae, preferably wherein the at least one bacteria include Lawsonella, Cutibacterium, or a combination thereof; and
(e) and determining the severity of the acne lesions based on the difference.

9. The method of claim 8, wherein the control is an amount of bacteria in a microbiome sample from a population of individuals not suffering from acne, wherein the bacteria is at least one bacteria selected from Lawsonella, Cutibacterium, Staphylococcus, Corynebacterium, Streptococcus, Micrococcus, and Neisseriaceae, preferably wherein the at least one bacteria is Lawsonella, Cutibacterium, or a combination thereof.

10. The method of claim 8, further comprising, treating the individual suffering from acne by minimizing the difference between amounts of the bacteria in the microbiome of the individual's skin and the control.

11. The method of claim 10, wherein the treatment comprises topically administering to the individual's skin a pharmaceutical or cosmetic composition that minimizes the difference between amounts of the bacterial in the microbiome of the individual's skin and the control.

12. The method of claim 11, wherein the pharmaceutical or cosmetic composition includes one or more anti-acne compounds selected from retinoids, anti-bacterial compounds, anti-inflammatory compounds, essential oils, a botanical extracts, probiotics, prebiotics, postbiotics, metabolites, vitamins, benzoyl peroxide, sulfur, or combinations thereof.

13. The method of claim 10, wherein the treatment comprising orally administering an active compound that minimizes the difference between the amount of the bacterial in the microbiome of the individual's skin and the control.

14. The method of claim 13, wherein the compound is a retinoid, an antibiotic, a hormone, a zinc salt, a prebiotic, postbiotics, metabolites, a probiotic, an omega-3-fatty acid, a vitamin, or combinations thereof.

15. A method for personalized prevention and treatment of acne comprising:
(a) obtaining microbiome sample from skin of an individual;
(b) determining an amount of one or more bacteria in the microbiome sample, wherein the one or more bacteria are selected from Lawsonella, Cutibacterium, Staphylococcus, Corynebacterium, Streptococcus, Micrococcus, and Neisseriaceae, preferably wherein the one or more bacteria include Lawsonella, Cutibacterium, or a combination thereof;
(c) comparing the amount of the one or more bacteria to a control;
(d) identifying a difference between the amount of the one or more bacteria in the microbiome sample and the control with respect to at least one bacteria selected from Lawsonella, Cutibacterium, Staphylococcus, Corynebacterium, Streptococcus, Micrococcus, and Neisseriaceae, preferably wherein the at least one bacteria include Lawsonella, Cutibacterium, or a combination thereof; and
(c) treating the individual by minimizing the difference between amounts of the one or more bacteria in the microbiome of the individual's skin and the control.

16. The method of claim 15, wherein the control is an amount of bacteria in a microbiome sample from a population of individuals not suffering from acne, wherein the bacteria is at least one bacteria selected from Lawsonella, Cutibacterium, Staphylococcus, Corynebacterium, Streptococcus, Micrococcus, and Neisseriaceae, preferably wherein the at least one bacteria is Lawsonella, Cutibacterium, or a combination thereof.

17. The method of claim 16, wherein the treatment comprises topically administering to the individual's skin a pharmaceutical or cosmetic composition that minimizes the difference between the amount of the bacterial in the microbiome of the individual's skin and the control.

18. The method of claim 17, wherein the pharmaceutical or cosmetic composition includes one or more anti-acne compounds selected from retinoids, anti-bacterial compounds, anti-inflammatory compounds, essential oils, a botanical extracts, probiotics, prebiotics, postbiotics, metabolites, vitamins, benzoyl peroxide, sulfur, or combinations thereof.

19. The method of claim 15, wherein the treatment comprising orally administering an active compound that minimizes the difference between the amount of the bacterial in the microbiome of the individual's skin and the control.

20. The method of claim 19, wherein the compound is a retinoid, an antibiotic, a hormone, a zinc salt, a prebiotic, a probiotic, an omega-3-fatty acid, a vitamin, or combinations thereof.
